(19) United States
(12) Patent Application Publication
(10) Pub. No.: US 2026/0272811 A1
(43) Pub. Date: Sep. 17, 2026

(54) DISPERSION STABILIZING AGENT

(71) Applicant: KENVUE BRANDS LLC, Summit, NJ (US)

(72) Inventors: Nadine CHERRAK, Agnadello (IT); Jeffrey D. MARTIN, Lawrence Twp., NJ (US)

(21) Appl. No.: 19/369,472

(22) Filed: Oct. 27, 2025

Related U.S. Application Data
(60) Provisional application No. 63/711,929, filed on Oct. 25, 2024.

(51) Int. Cl. A61K 8/73; A61K 8/04; A61K 8/06; A61Q 19/00

(52) U.S. Cl. CPC A61K 8/731; A61K 8/044; A61K 8/06; A61K 8/73; A61K 8/737; A61Q 19/00; A61K 2800/10; A61K 2800/52

(57) ABSTRACT

The invention generally relates to dispersion stabilizing agents comprising 65% or more of cellulose gum, 5% to 25% of carrageenan, 5% to 25% of carob gum, 0.1% to 4% of xanthan gum, and 0.1% to 4% of inulin, in wt % of the dispersion stabilizing agent; and to cosmetic formulations comprising said dispersion stabilizing agents. The invention also relates to methods of increasing the stability of cosmetic formulations such as emulsions or suspensions.

### PRIORITY CLAIM

The present application claims priority to U.S. Provisional Application Ser. No. 63/711,929 filed Oct. 25, 2024, the entire contents of which is hereby incorporated by reference herein.

### FIELD

The invention generally relates to dispersion stabilizing agents and to cosmetic formulations comprising said dispersion stabilizing agents. The invention also relates to methods of increasing the stability of cosmetic formulations such as emulsions or suspensions.

### BACKGROUND

Liquid dispersions such as emulsion or suspension are common among cosmetic formulations such as leave-on (creams, lotions) or rinse-off (body washes, shampoos). Although liquid dispersions are thermodynamically unstable, it is possible to extend their shelf life by extending their kinetical stability with dispersion stabilizing agents. Dispersion stabilizing agents known for use in cosmetic formulations include synthetic polymers such as carbomers (acrylic acid polymers) or natural polymers, such as cellulose gum, starch, or polysaccharide gums. Carbomers are water swellable polymers which provide desirable properties including, thickening, desirable feel, in addition to formulation stability.

Dispersion stabilizing agents contribute to the physical properties of cosmetic formulations which provide a pleasing sensory experience for the consumer. The physical properties that influence consumer experience include viscosity, yield stress, yield risk, and shear thinning. Synthetic polymers are well known to provide a desirable sensory experience, however some consumers may have a preference for cosmetic formulations that use natural ingredients.

Replacing synthetic polymer dispersion stabilizing agents in cosmetic formulations is challenging. Natural polymers can be inconsistent which causes variability in performance. As a result, finding natural polymers that will provide cosmetic formulations with the same or better consumer experience may require extensive experimentation. The challenges of replacing synthetic polymers with natural polymers are described in Tafuro et al., “As expected, natural-derived polysaccharides do not reach the mechanical properties of highly crosslinked acrylic polymers such as carbomer, which showed higher physical parameters. Matching the performance of synthetic polymers by replacing them with polysaccharides is difficult because each raw material has its own sensorial specificities and synthetic polymers are artificially created to have a specific performance in formulation” (Tafuro et al, Evaluating natural alternatives to synthetic acrylic polymers: rheological and texture analyses of polymeric water dispersions, ACS Omega 2020 5 (25), 15280-15289).

Moisturizers are mixtures of chemical agents specially designed to provide cosmetic benefits by enhancing the skin's surface layers appearance and moisturization levels. Cosmetic formulations with moisturizing properties are known in the personal care industry. Consumers expect such formulations to satisfy a range of requirements. Apart from the skin/hair-care benefits which determine the intended application, value is placed on such diverse parameters as dermatological safety, appearance, sensory attributes, shelf-life stability in storage and ease of use.

Therefore there is an ongoing need for cosmetic formulations based on liquid dispersions, such as emulsion or suspension, using natural dispersion stabilizing agents. There is an ongoing need for leave-on cosmetic formulations such as creams or lotions, based on liquid dispersions using natural dispersion stabilizing agents. There is an ongoing need for rinse-off cosmetic formulations such as body washes or shampoo, based on liquid dispersions using natural dispersion stabilizing agents.

Therefore there is an ongoing need for cosmetic formulations based on liquid dispersions, such as emulsion or suspension, which provide moisturization but also maintain other desirable properties using natural dispersion stabilizing agents. There is an ongoing need for dispersion stabilizing agents derived from renewable natural sources, for example for the stabilization of emulsions or suspensions.

### SUMMARY

One aspect of the disclosure pertains to a dispersion stabilizing agent, comprising: 65% or more of cellulose gum, 5% to 25% of carrageenan, 5% to 25% of carob gum, 0.1% to 4% of xanthan gum, and 0.1% to 4% of inulin, in wt % of the dispersion stabilizing agent.

This aspect may be combined with a variety of embodiments, in any combination. Thus, in some embodiments the dispersion stabilizing agent further comprises at least one carbohydrate selected from a group consisting of cellulose, glucose, fructose, or combinations thereof.

In some embodiments the dispersion stabilizing agent is a blend of a first premix and a second premix, wherein the first premix comprises: cellulose gum, carrageenan and carob gum, and the second premix comprises: cellulose gum, xanthan gum and inulin. In some embodiments the first premix comprises: 75% or more of cellulose gum, 10% to 15% of carrageenan; and 10% to 15% of carob gum, in wt % of the first premix. In some embodiments the second premix comprises: 50% or more of cellulose gum, 5% to 10% of xanthan gum, and 5% to 10% of inulin, in wt % of the second premix. In some embodiments the ratio between the first premix and the second premix is 2.1:1 to 10:1. In some embodiments the first premix comprise 75% or more of cellulose gum, 10% to 15% of carrageenan; and 10% to 15% of carob gum, in wt % of the first premix, and the second premix comprise 50% or more of cellulose gum, 5% to 10% of xanthan gum, and 5% to 10% of inulin, in wt % of the second premix, and the ratio between the first premix and the second premix is 2.1:1 to 10:1.

Another aspect of the disclosure pertains to a cosmetic formulation comprising the dispersion stabilizing agent according to any of the embodiments described above. This aspect may be combined with a variety of embodiments, in any combination. Thus, in some embodiments the dispersion stabilizing agent is a blend of a first premix and a second premix and wherein the first premix comprises: cellulose gum, carrageenan and carob gum, and the second premix comprises: cellulose gum, xanthan gum and inulin. In some embodiments of the cosmetic formulation the amount of dispersion stabilizing agent is from 0.1% or more to 5% or less, in wt % of the cosmetic formulation. In some embodiments the formulation has a yield stress of more than 30 Pa. In some embodiments the formulation is an emulsion or a suspension.

Another aspect of the disclosure pertains to a method of increasing the stability of a cosmetic emulsion, comprising the combination of the dispersion stabilizing agent, according to any of the embodiments described above, with a cosmetic emulsion.

Another aspect of the disclosure pertains to a method of increasing the stability of a cosmetic suspension, comprising the combination of the dispersion stabilizing agent, according to any of the embodiments described above, with a cosmetic suspension.

### DETAILED DESCRIPTION

As used herein, a “dispersion” is an heterogenous mixture of solid particles or liquid droplets in a continuous liquid phase (also called “matrix phase”). The solid particles or a liquid droplet are not soluble (not dissolvable, not miscible) in the continuous liquid phase. The continuous liquid phase may be aqueous, for example water, or non-aqueous, for example an oil; in the field of cosmetic formulations the continuous liquid phase is preferably water, but in specific formulations it can be an oil. “Heterogenous mixture” means that it is possible to distinguish the constituents at least under a microscope.

As used herein, a “premix” is a pre-prepared mixture of various ingredients that has been mixed in advance of use or of further processing.

As used herein, “yield stress” is a measure of the behavior of a formulation and how it deforms under stress. Yield stress values give an indication about the long-term stability of the cosmetic formulation, but also on the usability of the formulation, for example its pumpability (further depending on the nature of the pump used) and its behavior when rubbed on the skin (spreadability).

As used herein “long term” means several months and preferably several years, for example 1, 2, 3, 4, 5 or more years.

As used herein “spreadability” means the behavior of a cosmetic formulation when spread on the skin, the capability of the cosmetic formulation to cover an area of the skin, and the viscosity behavior of the cosmetic formulation while it is being spread on the skin.

As used herein, “yield risk” is a measure of the liquid-like or solid-like behavior of a cosmetic formulation. Yield risk values give an indication about the yield stress at the deformation conditions of interest, and the robustness of the cosmetic formulation.

As used herein, “flow break”, or shear thinning index, is a measure of the decrease of viscosity with increasing shear rate. Flow break values give an indication about the behavior of the cosmetic formulation when rubbed on the skin, for example the ease of spreadability.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. The general convention in the scientific and technical literature is applied: the last decimal place of a numerical value indicates its degree of accuracy. Where no other error margins are given, the maximum margin is ascertained by applying the rounding-off convention to the last decimal place, for example for a measurement of 3.5%, the error margin is 3.45-3.54.

All percentages (%) are by weight (wt %) unless otherwise specified herein.

As used herein a “natural dispersion stabilizing agent” is a dispersion stabilizing agent derived from a renewable natural source such as plants, algae, bacteria, insects or other animals or any other living organisms.

One aspect of the disclosure pertains to a dispersion stabilizing agent, comprising:
65% or more of cellulose gum,
5% to 25% of carrageenan,
5% to 25% of carob gum,
0.1% to 4% of xanthan gum, and
0.1% to 4% of inulin,
in wt % of the dispersion stabilizing agent.

Surprisingly, it has been discovered that a mixture of natural ingredients such as natural gum carbohydrates and polysaccharides according to the disclosure could provide a dispersion stabilizing agent with stabilization increasing properties and viscosity increasing properties similar to synthetic polymers such as carbomer.

A dispersion stabilizing effect on a formulation may be measured typically by the yield stress value. A yield stress value above or equal to 30 Pa may be considered sufficient to provide stability to a cosmetic formulation, such as an emulsion or a suspension. In other words, a yield stress value above or equal to 30 Pa may be desirable to ensure the oily phase micelles, in the case of an emulsion, or the solid particles, in the case of a suspension, remain homogenously dispersed in the aqueous phase of the formulation.

In another embodiment the dispersion stabilizing agent, may comprise:
70% or more of cellulose gum,
7% to 12% of carrageenan,
7% to 12% of carob gum,
0.5% to 3% of xanthan gum, and
0.5% to 3% of inulin,
in wt % of the dispersion stabilizing agent.

In one or more embodiments, the dispersion stabilizing agent may further comprises at least one carbohydrate selected from a group consisting of cellulose, glucose, fructose, or combinations thereof.

In one or more embodiments, the dispersion stabilizing agent may further comprises 1.6% or less of cellulose.

In one or more embodiments, the dispersion stabilizing agent may further comprises 1.6% or less of fructose.

In one or more embodiments, the dispersion stabilizing agent may further comprises 5% or less of glucose.

In one or more embodiments, the dispersion stabilizing agent may further comprises 1.6% or less of cellulose, 1.6% or less of fructose and 5% or less of glucose.

In one or more embodiments the dispersion stabilizing agent, may comprise:
70% or more of cellulose gum,
7% to 12% of carrageenan,
7% to 12% of carob gum,
0.5% to 3% of xanthan gum,
0.5% to 3% of inulin,
1.6% or less of cellulose,
1.6% or less of fructose, and
5% or less of glucose.
in wt % of the dispersion stabilizing agent.

In one or more embodiments the dispersion stabilizing agent may be a blend of a first premix and a second premix, and wherein
the first premix comprises: cellulose gum, carrageenan and carob gum, and
the second premix comprises: cellulose gum, xanthan gum and inulin.

It may be more convenient to use premixes of already blended raw materials to limit the number of materials to handle in the context of industrial manufacturing. It has been found that a combination of two premixes provides the desirable simplicity.

Surprisingly, it has been discovered that a blend of the first and second premixes according to the disclosure provides a dispersion stabilizing agent despite each premix having no effect on viscosity when taken individually. In other words, the first premix alone in water does not increase the yield stress of the aqueous formulation, the second premix alone in water does not increase the yield stress of the aqueous formulation; but when combined in water the first and second premixes have an increasing effect on the yield stress of the water formulation.

A dispersion stabilizing effect on a formulation may be measured typically by the yield stress value. A yield stress value above or equal to 30 Pa may be considered sufficient to provide stability to a cosmetic formulation, such as an emulsion or a suspension. In other words, a yield stress value above or equal to 30 Pa may be desirable to ensure the oily phase micelles, in the case of an emulsion, or the solid particles, in the case of a suspension, remain homogenously dispersed in the aqueous phase of the formulation.

In one or more embodiments, the first premix may comprise 75% or more of cellulose gum, in wt % of the first premix.

In one or more embodiments, the first premix may comprise 10% to 15% of carrageenan, in wt % of the first premix.

In one or more embodiments, the first premix may comprise 10% to 15% of carob gum, in wt % of the first premix.

In one or more embodiments, the first premix may comprise:
75% or more of cellulose gum,
10% to 15% of carrageenan, and
10% to 15% of carob gum, in wt % of the first premix.

In one or more embodiments, the cellulose gum may be a cellulose carboxymethyl ether sodium salt.

In one or more embodiments, the second premix may comprise 50% or more of cellulose gum, in wt % of the second premix.

In one or more embodiments, the second premix may comprise 5% to 10% of xanthan gum, in wt % of the second premix.

In one or more embodiments, the second premix may comprise 5% to 10% of inulin, in wt % of the second premix.

In one or more embodiments, the second premix may comprise:
50% or more of cellulose gum,
5% to 10% of xanthan gum, and
5% to 10% of inulin, in wt % of the second premix.

In one or more embodiments, the ratio between the first premix and the second premix may be 2.1:1 to 10:1.

The ratio between the first premix and the second premix may be 2.2:1 to 7:1. The ratio between the first premix and the second premix may be 2.3:1 to 6:1. The ratio between the first premix and the second premix may be 2.5:1 to 5:1. The ratio between the first premix and the second premix may be 2.1:1 to 7:1. The ratio between the first premix and the second premix may be 2.1:1 to 6:1. The ratio between the first premix and the second premix may be 2.1:1 to 5:1. The ratio between the first premix and the second premix may be 3:1 to 6:1.

In one or more embodiments, the disclosure pertains to a dispersion stabilizing agent, comprising a blend of a first premix and a second premix, wherein
the first premix may comprise 75% or more of cellulose gum, 10% to 15% of carrageenan, and 10% to 15% of carob gum, in wt % of the first premix, and
the second premix may comprise 50% or more of cellulose gum, 5% to 10% of xanthan gum, and 5% to 10% of inulin, in wt % of the second premix, and the ratio between the first premix and the second premix may be 2.1:1 to 10:1, or 2.3:1 to 6:1, or 2.5:1 to 5:1, or 3:1 to 6:1.

In one or more embodiments, the disclosure pertains to a dispersion stabilizing agent, comprising a blend of a first premix and a second premix, wherein
the first premix may comprise 75% or more of cellulose gum, 10% to 15% of carrageenan, and 10% to 15% of carob gum, in wt % of the first premix, and
the second premix may comprise 50% or more of cellulose gum, 5% to 10% of xanthan gum, and 5% to 10% of inulin, in wt % of the second premix, and the dispersion stabilizing agent may further comprise at least one carbohydrate selected from a group consisting of cellulose, glucose, fructose, or combinations thereof; for example 1.6% or less of cellulose, 1.6% or less of fructose and 5% or less of glucose; and wherein the ratio between the first premix and the second premix may be 2.1:1 to 10:1, or 2.3:1 to 6:1, or 2.5:1 to 5:1, or 3:1 to 6:1.

In another aspect, the disclosure pertains to a cosmetic formulation comprising a dispersion stabilizing agent according to the present disclosure.

In one or more embodiments, the cosmetic formulation may further comprise a solvent. Any suitable solvent may be used in the cosmetic formulation as the context dictates. In some embodiments, the solvent is a dermatologically acceptable solvent. As will be recognized by those of skill in the art, dermatologically acceptable solvents comprise solvents that are suitable for use in contact with the body, in particular the skin, without undue toxicity, incompatibility, instability, irritation, allergic response, and the like. A safe and effective amount of dermatologically acceptable solvent is from about 50%, 55%, 60%, 65%, 70%, 75%, 80%, 85%, 90%, 95%, or 98% to about 85%, 90%, 95%, 98%, 99%, 99.1%, 99.5% or 99.9% in wt % of the cosmetic formulation.

The following are non-limiting examples of dermatologically acceptable solvents. Other dermatologically acceptable solvents can be formulated by those of ordinary skill in the art. In one embodiment, the solvent may contain water.

In a further embodiment, the cosmetic formulation may also comprise one or more aqueous or organic solvents. Examples of organic solvents include, but are not limited to dimethyl isosorbide, isopropyl myristate, vegetable oils, mineral oils, alkanols, glycols, and polyols. Examples of glycols include, but are not limited to, glycerin, propylene glycol, butylene glycol, pentalene glycol, hexylene glycol, polyethylene glycol, polypropylene glycol, diethylene glycol, triethylene glycol, capryl glycol, glycerol, butanediol, hexanetriol, and copolymers or mixtures thereof. Examples of alkanols include, but are not limited to, those having from about 2 carbon atoms to about 12 carbon atoms (e.g., from about 2 carbon atoms to about 4 carbon atoms), such as isopropanol and ethanol. Examples of polyols include, but are not limited to, those having from about 2 carbon atoms to about 15 carbon atoms (e.g., from about 2 carbon atoms to about 10 carbon atoms), such as propylene glycol.

The organic solvents may be present in the cosmetic formulation in an amount of from about 1% to about 99.99% (e.g., from about 20% to about 50%), in wt % of the cosmetic formulation.

In one or more embodiments water may be present in the cosmetic formulation in an amount of from about 5% to about 95%, from about 40% to about 99.99%, from about 50% to about 99.9%, from about 60% to about 99%, from about 70% to about 99%, from about 70% to about 90%, or from about 70% to 85% of water, in wt % of the cosmetic formulation.

In one or more embodiments, the cosmetic formulation may further comprise at least one emollient. The cosmetic formulation may comprise from about 1% to about 20%, from about 1% to about 10%, from about 1% to about 5%, from about 5% to about 20%, from about 5% to about 15%, from about 5% to about 10%, from about 10% to about 20%, from about 10% to about 15%, of at least one emollient, in wt % of the cosmetic formulation.

What is meant by an emollient is a compound that helps to maintain the soft, smooth, and pliable appearance of the skin (e.g., by remaining on the skin surface or in the stratum corneum to act as a lubricant). Examples of suitable emollients include, but are not limited to, petrolatum, isopropyl palmitate, vegetable oils whether or not hydrogenated, silicone oils, any liquid polymerized siloxane, such as dimethicone, cyclomethicone, cyclopentasiloxane, diméthiconol and amodimethicone, mineral oil, waxes such as parrafin, paraffinum liquidum, and petrolatum, aloe vera, fatty alcohols such as arachidyl alcohol, batyl alcohol, behenyl alcohol, C12-13 alcohols, cetearyl alcohol, cetyl alcohol, coconut alcohol, isocetyl alcohol, octyldodecanol and palm alcohol, fatty acids and esters of fatty acids such as batyl stearate, cetearyl behenate, cetearyl isononanoate, cetyl esters, coconut acid, ethyl linoleate, ethyl linolenate, ethyl olivate, ethylhexyl cocoate, ethylhexyl myristate, glyceryl caprylate, glyceryl laurate/oleate, glyceryl ricinoleate, isocetyl salicylate, isostearyl alcohol, linoleic acid, linolenic acid, oleic acid, palm kernel acid, palmitic acid and wheat germ acid, shea butter (Butyrospermum parkii butter), cocoa butter (Theobroma cacao butter), waxes, derivatives of glycerides such as capric/caprylic triglyceride, cocoglycerides, and palm glycerides, cholesterol, lanolin and derivatives, derivatives of PEGs, squalane, squalene, sucrose and derivatives, glycerol derivatives such as glycerol trioctanoate (tricaprylin), glycerol, tristearate (tristearin), and mixtures thereof.

In one or more embodiments, the cosmetic formulation may further comprise at least one emulsifier. The cosmetic formulation may comprise from about 0.1% to about 10%, from about 0.1% to about 5%, from about 0.1% to about 2%, from about 0.1% to about 1%, about 0.5% to about 10%, from about 0.5% to about 5%, from about 0.5% to about 2%, from about 0.5% to about 1%, about 1% to about 10%, from about 1% to about 5%, from about 1% to about 2%, in wt % of the cosmetic formulation.

Emulsifiers are surfactants that stabilize an emulsion by decreasing the surface tension between the oil and the water phases. Emulsifiers may be nonionic, amphoteric, anionic or cationic.

A variety of other dermatologically acceptable materials may also be present in the cosmetic formulation of the present disclosure. In one or more embodiments, the cosmetic formulation may comprise one or more topical ingredients selected from a group consisting of, surfactants, chelating agents, humectants, conditioners, preservatives, opacifiers, fragrances and the like.

What is meant by a humectant is a compound intended to increase the water content of the top layers of skin (e.g., hygroscopic compounds). Examples of suitable humectants include, but are not limited to, glycerin, sorbitol or trehalose (e.g., α,α-trehalose, β,β-trehalose, α,β-trehalose) or a salt or ester thereof (e.g., trehalose 6-phosphate).

What is meant by a surfactant is a surface-active agent intended to cleanse or emulsify. Examples of suitable surfactants include anionic surfactants, cationic surfactants, amphoteric surfactants such as betaines, nonionic surfactants such as alkyl polyglucosides.

Examples of suitable dermatologically acceptable chelating agents include those which are capable of protecting and preserving the cosmetic formulation of the present disclosure. In one or more embodiments, the chelating agent is ethylenediaminetetraacetic acid (“EDTA”), and more specifically is tetrasodium EDTA, available commercially from Dow Chemical Company of Midland, Michigan under the trade name, “Versene 100XL.”

Suitable dermatologically acceptable preservatives include, for example, parabens, quaternary ammonium species, phenoxyethanol, benzoates, DMDM hydantoin, organic acids and may be present in the cosmetic formulation in an amount from about 0% to about 1% or from about 0.05% to about 0.5%, in wt % of the cosmetic formulation.

Any of a variety of conditioners which impart additional attributes, such as gloss to the hair, are suitable for use in cosmetic formulation of the present disclosure. Examples include, but are not limited to, volatile silicone conditioning agent having an atmospheric pressure boiling point less than about 220° C. Examples of suitable volatile silicones nonexclusively include polydimethylsiloxane, polydimethylcyclosiloxane, hexamethyldisiloxane, cyclomethicone fluids such as polydimethylcyclosiloxane available commercially from Dow Corning Corporation of Midland, Michigan under the tradename, “DC-345” and mixtures thereof, and specifically include cyclomethicone fluids. Other suitable conditioners include cationic polymers, including polyquarterniums, cationic guar, and the like.

Any of a variety of commercially available pearlescent or opacifying agents are suitable for use in the cosmetic formulation of the present disclosure. Examples of suitable pearlescent or opacifying agents include, but are not limited to, mono or diesters of (a) fatty acids having from about 16 to about 22 carbon atoms and (b) either ethylene or propylene glycol; mono or diesters of (a) fatty acids having from about 16 to about 22 carbon atoms (b) a polyalkylene glycol of the formula: HO-(JO)a-H, wherein J is an alkylene group having from about 2 to about 3 carbon atoms; and a is 2 or 3; fatty alcohols containing from about 16 to about 22 carbon atoms; fatty esters of the formula: KCOOCH2L, wherein K and L independently contain from about 15 to about 21 carbon atoms; inorganic solids insoluble in a shampoo formulation, and mixtures thereof.

Any fragrance suitable for use on skin may be used in the cosmetic formulation according to the present disclosure.

In one or more embodiments of the cosmetic formulation, the dispersion stabilizing agent may comprise a blend of a first premix and a second premix wherein
the first premix comprises: cellulose gum, carrageenan and carob gum, and
the second premix comprises: cellulose gum, xanthan gum and inulin.

In one or more embodiments of the cosmetic formulation, the dispersion stabilizing agent may be a blend of a first premix and a second premix wherein
the first premix comprises: cellulose gum, carrageenan and carob gum, and
the second premix comprises: cellulose gum, xanthan gum and inulin.

In one or more embodiments of the cosmetic formulation, the amount of the first premix may be from 1% to 5%, in wt % of the cosmetic formulation.

In one or more embodiments of the cosmetic formulation, the amount of the first premix may be 1% or more or 1.5% or more, or 2% or more, in wt % of the cosmetic formulation.

In one or more embodiments of the cosmetic formulation, the amount of the first premix may be 5% or less, or 4% or less, or 3% or less, in wt % of the cosmetic formulation.

In one or more embodiments of the cosmetic formulation, the amount of the first premix may be from 2% to 3%, or preferably about 2.5%, in wt % of the cosmetic formulation.

In one or more embodiments of the cosmetic formulation, the amount of the second premix may be from 0.1% to 2.5%, in wt % of the cosmetic formulation.

In one or more embodiments of the cosmetic formulation, the amount of the second premix may be 0.1% or more, or 0.2% or more, or 0.3% or more, in wt % of the cosmetic formulation.

In one or more embodiments of the cosmetic formulation, the amount of the second premix may be 2.5% or less, or 2% or less, or 1.5% or less, in wt % of the cosmetic formulation.

In one or more embodiments of the cosmetic formulation, the amount of the second premix may be from 0.3% to 1.5%, or preferably from 0.5% to 1%, in wt % of the cosmetic formulation.

In one or more embodiments of the cosmetic formulation, the dispersion stabilizing agent may be a blend of a first premix and a second premix wherein
the first premix comprises: cellulose gum, carrageenan and carob gum, and the amount of the first premix is from 1% to 5%, in wt % of the cosmetic formulation, and
the second premix comprises: cellulose gum, xanthan gum and inulin, and the amount of the second premix is from 0.1% to 2.5%, in wt % of the cosmetic formulation.

In one or more embodiments of the cosmetic formulation, the dispersion stabilizing agent may be a blend of a first premix and a second premix wherein
the first premix comprises: cellulose gum, carrageenan and carob gum, and the amount of the first premix is from 2% to 3%, in wt % of the cosmetic formulation, and
the second premix comprises: cellulose gum, xanthan gum and inulin, and the amount of the second premix is from 0.5% to 1%, in wt % of the cosmetic formulation.

In one or more embodiments of the cosmetic formulation, the dispersion stabilizing agent may be a blend of a first premix and a second premix wherein
the first premix may comprise 75% or more of cellulose gum, 10% to 15% of carrageenan, and 10% to 15% of carob gum, in wt % of the first premix, and
the second premix may comprise 50% or more of cellulose gum, 5% to 10% of xanthan gum, and 5% to 10% of inulin, in wt % of the second premix, and the ratio between the first premix and the second premix may be 2.1:1 to 10:1, or 2.3:1 to 6:1, or 2.5:1 to 5:1, or 3:1 to 6:1.

In one or more embodiments of the cosmetic formulation, the dispersion stabilizing agent may be a blend of a first premix and a second premix wherein
the first premix may comprise 75% or more of cellulose gum, 10% to 15% of carrageenan, and 10% to 15% of carob gum, in wt % of the first premix, and
the second premix may comprise 50% or more of cellulose gum, 5% to 10% of xanthan gum, and 5% to 10% of inulin, in wt % of the second premix, and the dispersion stabilizing agent may further comprise at least one ingredient selected from a group consisting of cellulose, glucose, fructose, or combinations thereof; for example 1.6% or less of cellulose, 1.6% or less of fructose and 5% or less of glucose; and wherein the ratio between the first premix and the second premix may be 2.1:1 to 10:1, or 2.3:1 to 6:1, or 2.5:1 to 5:1, or 3:1 to 6:1.

In one or more embodiments, the cosmetic formulation may further comprise Sodium Polyacrylate. Sodium Polyacrylate may be present in more than 0.01%, more than 0.05%, more than 0.1%, more than 0.2%, in wt % of the cosmetic formulation. Sodium Polyacrylate may be present in less than 5%, less than 2%, less than 1%, in wt % of the cosmetic formulation. Preferably the amount of Sodium Polyacrylate may be from 0.01% to 5%, from 0.05% to 2%, preferably from 0.1% to 1%, preferably about 0.5%, in wt % of the cosmetic formulation.

In one or more embodiments, the cosmetic formulation may comprise an amount of the dispersion stabilizing agent from 0.1% or more to 5% or less, in wt % of the cosmetic formulation.

In one or more embodiments, the cosmetic formulation may comprise an amount of the dispersion stabilizing agent from 0.5% or more to 4% or less, in wt % of the cosmetic formulation.

In one or more embodiments, the cosmetic formulation may comprise an amount of the dispersion stabilizing agent from 1% or more to 4% or less, in wt % of the cosmetic formulation.

In one or more embodiments, the cosmetic formulation may comprise an amount of the dispersion stabilizing agent from 1.5% or more to 4% or less, in wt % of the cosmetic formulation.

In one or more embodiments, the cosmetic formulation may comprise an amount of the dispersion stabilizing agent from 0.5% or more to 3% or less, in wt % of the cosmetic formulation.

In one or more embodiments, the cosmetic formulation may comprise an amount of the dispersion stabilizing agent from 1.5% or more to 3% or less, in wt % of the cosmetic formulation.

In one or more embodiments, the cosmetic formulation may have a yield stress of more than 30 Pa, preferably of more than 35 Pa.

In one or more embodiments, the cosmetic formulation may have a yield stress of less than 250 Pa, preferably of less than 150 Pa, preferably of less than 100 Pa.

To impart stability, the yield stress must be large enough to counteract buoyancy forces but not so large that the usability of the formula is compromised, e.g., pumpability and spreadability.

In one or more embodiments, the cosmetic formulation may have a yield risk of less than 1, preferably less than 0.9, preferably less than 0.8.

Yield risk (or tan(delta)) can range from zero (a perfect Hookean solid) to infinity (a perfect Newtonian liquid), with the transition from solid-like to liquid-like dominance occurring at a value of one. Thus, Yield risk must be lower than one for a material to be solid-dominated and consequently have a yield stress at the deformation conditions of interest, with the robustness of the yield condition increasing the further below one Yield risk becomes.

In one or more embodiments, the cosmetic formulation may have a shear thinning index (or Flow/break) of more than 0.6, or more than 0.7, preferably more than 0.8.

The shear thinning index, n, can range from zero (no shear thinning) to 1 (instant shear thinning). Highly shear thinning behavior leads to ease of spreadability and “break”, where the formula transitions from a solid while resting on the skin to a shear-thinning liquid while it is being rubbed into the skin. Ease of spreadability and quick break contribute to desirable aesthetics of cosmetic and personal care products.

In one or more embodiments, the cosmetic formulation may be an emulsion or a suspension.

As used herein, an “emulsion” is a mixture of two or more liquids that are normally immiscible. Examples of emulsions, include, but are not limited to: oil-in-water, water-in-oil, water-in-oil-in-water, and oil-in-water-in-silicone emulsions. These emulsions can cover a broad range of viscosities, for example, from about 100 cps to about 200,000 cps using a Brookfield RVT viscometer.

As used herein, a “suspension” is a heterogeneous mixture of a liquid phase that contains non dissolvable solid particles.

In one or more embodiments, the cosmetic formulation may be an emulsion comprising a dispersion stabilizing agent according to the present disclosure, wherein
the amount of the first premix is from 1% to 5%, and
the amount of the second premix is from 0.1% to 2.5%, in wt % of the cosmetic formulation.

In one or more embodiments, the cosmetic formulation may be a suspension comprising a dispersion stabilizing agent according to the present disclosure, wherein
the amount of the first premix is from 1% to 5%, and
the amount of the second premix is from 0.1% to 2.5%, in wt % of the cosmetic formulation.

In another aspect, the disclosure pertains to a method for increasing the stability of a cosmetic emulsion, wherein the method comprises the step of combining the dispersion stabilizing agent according to the present disclosure, with a cosmetic emulsion.

As used herein, by “cosmetic emulsion” it is meant a cosmetic formulation which is an emulsion.

As used herein, by “stability” of the cosmetic emulsion, it is meant that the emulsion will remain stable and resist changes such as flocculation, coalescence, creaming or sedimentation, changes in pH and changes in appearance such as color, texture, odor, when stored in a protective packaging, for a period of time of at least 1 year, preferably 2 years, under normal conditions such as 30° C. and 75% relative humidity.

In another aspect, the disclosure may pertain to the use of the dispersion stabilizing agent according to the present disclosure, in a cosmetic emulsion, to increase the stability of said emulsion.

In another aspect, the disclosure pertains to a method for increasing the stability of a cosmetic suspension, wherein the method comprises the step of combining the dispersion stabilizing agent according to the present disclosure, with a cosmetic suspension.

As used herein, by “cosmetic suspension” it is meant a cosmetic formulation which is a suspension.

As used herein, by “stability” of the cosmetic suspension, it is meant that the suspension will remain stable and resist changes such as phases separation (flocculation, aggregation or sedimentation), changes in pH and changes in appearance such as color, texture, odor, when stored in a protective packaging, for a period of time of at least 1 year, preferably 2 years, under normal conditions such as 30° C. and 75% relative humidity.

In another aspect, the disclosure may pertain to the use of the dispersion stabilizing agent according to the present disclosure, in a cosmetic suspension, to increase the stability of said suspension.

While the foregoing description represent exemplary embodiments of the present invention, it will be understood that various additions, modifications and substitutions may be made without departing from the spirit and scope of the present invention. In particular, it will be clear to those skilled in the art that the present invention may be embodied in other specific forms, structures, arrangements, proportions, and with other elements, materials, and components, without departing from the spirit or essential characteristics thereof. One skilled in the art will appreciate that the invention may be used with many modifications of structure, arrangement, proportions, materials, and components and otherwise, used in the practice of the invention, which are particularly adapted to specific environments and operative requirements without departing from the principles of the present invention. The presently disclosed embodiments are therefore to be considered in all respects as illustrative and not restrictive, the scope of the invention being indicated by the appended claims, and not limited to the foregoing description.

The present invention may be better understood through the following examples, which are exemplary in nature and not intended to be limiting to any specific combination of elements.

### EXAMPLES

### Materials

Carbomer; supplied by Lubrizol Beauty and Personal Care under the tradename “Carbopol Ultrez10”.
Dehydroxanthan Gum; supplied by Nouryon Chemicals Holding B.V. (formerly AkzoNobel Specialty Chemicals) under the tradename “Amaze XT Polymer”.
Hydroxypropyl Methylcellulose (and) Cellulose Gum (and) Xanthan Gum; supplied by Ashland Inc under the tradename “Texturpure”.
Cellulose Gum (and) Carrageenan (and) Ceratonia siliqua (Carob) Gum (and) Glucose; supplied by Omya Inc under the tradename “Sucraclear V2”. Formulation of this premix is: Cellulose Gum more than 75%, Carrageenan from 10% to 24.9%, Ceratonia siliqua (Carob) Gum from 10% to 24.9%, Glucose from 1% to 4.9%; in wt % of the premix.
Cellulose Gum (and) Xanthan Gum (and) Inulin (and) Cellulose (and) Glucose (and) Fructose; supplied by Ultra Chemical Inc under the tradename “preBIULIN C90”. Formulation of this premix is: Cellulose Gum more than 50%, Xanthan Gum from 5% to 10%, Inulin from 5% to 10%, Cellulose less than 5%, Glucose less than 5%, Fructose less than 5%; in wt % of the premix.

### Viscosity Measurement Method

All experiments were performed on an Anton Paar MCR302 rheometer with a 1 degree cone and plate geometry that has been roughened to reduce slip. All experiments were performed at 25° C. using a Peltier element for temperature control.

### Yield Stress:

Yield stress is essential for long-term product stability. In a formulation with an immiscible phase dispersed in a matrix phase, the phases often differ in density which leads to creaming/sedimentation due to buoyancy forces. Imparting the matrix phase with a yield stress can suppress creaming/sedimentation, provided the yield is of sufficient magnitude to resist the buoyancy forces. The yield stress is typically created in the matrix phase by using polymers which provide yield either by intermolecular association, by swelling and physical jamming, or a combination of both. To impart stability, the yield stress must be large enough to counteract buoyancy forces but not so large that the usability of the formula is compromised, e.g., pumpability and spreadability.

Yield stress was determined as the oscillatory stress where the storage and loss moduli (G′, G″, respectively) cross in a strain sweep performed logarithmically from 0.1 to 1000% strain at a frequency of 1 rad/s.

### Yield Risk (Tan(Delta)):

All materials exhibit viscoelasticity, meaning they exhibit a mixture of liquid-like and solid-like behavior, the exact nature of which depends on the applied deformation. In order for a material to exhibit a yield stress, it must behave predominantly like a solid. Materials which exhibit liquid-dominated behavior under certain deformation conditions will flow indefinitely; conversely, materials which exhibit solid-dominated behavior under certain deformation conditions will resist flow and deform elastically. It logically follows that only materials which exhibit solid-dominated behavior under a given set of deformation conditions can be said to exhibit a yield stress.

A quantity that describes the nature of viscoelasticity under small, linear oscillatory deformations is tan(delta), the tangent of the phase shift angle delta, which is defined as the ratio of the loss modulus to the storage modulus, G″/G′. Tan(delta) was taken at a value of 0.1 rad/s in a frequency sweep which was performed logarithmically from 0.1 to 100 rad/s at a strain that resides in the linear viscoelastic region for each individual sample. A low frequency was chosen to correspond to relatively long timescales which correspond to that of sedimentation/creaming.

Tan(delta) can range from zero (a perfect Hookean solid) to infinity (a perfect Newtonian liquid), with the transition from solid-like to liquid-like dominance occurring at a value of one. Thus, tan(delta) must be lower than one for a material to be solid-dominated and consequently have a yield stress at the deformation conditions of interest, with the robustness of the yield condition increasing the further below one tan(delta) becomes.

### Flow/Break (Shear Thinning Index):

Shear thinning is the decrease of viscosity with increasing shear rate. Shear thinning occurs due to many reasons, and physically arises from viscosity-imparting constituents of the material and how they interact with increasing shear rate. Highly shear thinning behavior leads to ease of spreadability and “break”, where the formula transitions from a solid while resting on the skin to a shear-thinning liquid while it is being rubbed into the skin. Ease of spreadability and quick break contribute to desirable aesthetics of cosmetic and personal care products.

To quantify shear thinning and flow/break, a flow sweep was performed by logarithmically sweeping the shear rate from 0.0001 to 1,000 s-1 at 5 seconds per point. To evaluate the shear thinning index, we first noted the stress at which the viscosity begins to decrease from a low-stress constant plateau value in a plot of viscosity vs. shear stress. The portion of the flow curve beginning at this stress and ending at 2× this stress was fitted to a power law equation: shear stress=k*(shear rate)\hat{ }n, where k is a consistency and n is the shear thinning index for this portion of the curve which represents the flow/break region. The shear thinning index, n, can range from zero (no shear thinning) to 1 (instant shear thinning).

Polymers which impart yield and viscosity through inter (and sometimes intra) molecular associations, e.g., gums and polysaccharides, typically have shear thinning indices that are relatively low, meaning that viscosity decreases at a relatively low rate with increasing shear rate or shear stress. This is due to the nature of the polymers themselves; having functional groups that associate, there are always associations, which resist the shear thinning, occurring at the molecular level as the polymers flow past themselves and other polymer molecules. Conversely, polymers which impart yield and viscosity by creating a physically-jammed network, e.g., polyacrylates, typically have relatively high shear thinning indices. This is due to the fact that physically-jammed networks tends to flow in an avalanche-like manner once the yield stress is surpassed, with minimal intermolecular association as the polymer domains flow past each other.

### Physical Stability

Physical stability of cosmetic formulations was used as a screening tool to select the most stable formulations for long term performance at shelf. For semisolid dosage forms, such as oil in water emulsions for lotions or creams, samples were stressed at different conditions; typically at 40° C.; 50° C. for 1 to 12 week periods. Freeze/thaw cycles were applied. The attributes that were customarily monitored at each interval are: appearance, color, texture, odor, viscosity and pH.

### Example 1: Screening Dispersion Stabilizing Agents in Water

A selection of commercially available dispersion stabilizing agents derived from natural sources were evaluated and compared to a synthetic Carbomer. The selection criteria was the yield stress value of the aqueous formulation obtained when the dispersion stabilizing agent is dissolved in water. The objective of this first screening step was to identify the natural dispersion stabilizing agents having a capacity to stabilize an emulsion or a suspension. Results are presented in Table 1; % indicate the wt % of each dispersion stabilizing agent in water.

TABLE 1
Dispersion stabilizing agents formulations in water

| | | | Hydroxypropyl | Cellulose Gum (and) | Cellulose Gum (and) | | | |
|---|---|---|---|---|---|---|---|---|
| | | | Methylcellulose | Carrageenan (and) | Xanthan Gum (and) | | | |
| | | Dehydro | (and) Cellulose | Ceratonia Siliqua | Inulin (and) Cellulose | Yield | | |
| | | xanthan | Gum (and) | (Carob) Gum | (and) Glucose (and) | stress | Yield | Flow/ |
| | Carbomer | Gum | Xanthan Gum | (and) Glucose | Fructose | (Pa) | risk | break |
| Trial 1 | 0.30% | — | — | — | — | 75.2 | 0.043 | 0.921 |
| (comp.) | | | | | | | | |
| Trial 2 | — | 1.00% | — | — | — | 16.7 | 0.238 | 0.934 |
| (comp.) | | | | | | | | |
| Trial 3 | — | — | 1.00% | — | — | 7.2 | 0.533 | 0.357 |
| (comp.) | | | | | | | | |
| Trial 4 | — | — | — | 2.50% | — | 0 | 1.714 | 0.460 |
| (comp.) | | | | | | | | |
| Trial 5 | — | — | — | — | 2.50% | 0 | 1.747 | 0.439 |
| (comp.) | | | | | | | | |
| Trial 6 | — | 0.30% | — | 2.50% | — | 0 | 1.222 | 0.628 |
| (comp.) | | | | | | | | |
| Trial 7 | — | 0.20% | — | — | 2.50% | 0 | 1.473 | 0.567 |
| (comp.) | | | | | | | | |
| Trial 8 | — | — | 0.20% | 2.50% | — | 0 | 1.393 | 0.586 |
| (comp.) | | | | | | | | |
| Trial 9 | — | — | — | 0.50% | 2.50% | 0 | 1.223 | 0.664 |
| (comp.) | | | | | | | | |
| Trial 10 | — | — | — | 2.50% | 1% | 118.2 | 0.756 | 0.807 |
| (inv.) | | | | | | | | |

“comp.” means comparative; “inv.” means inventive.

Dehydroxanthan Gum and the premix Hydroxypropyl Methylcellulose (and) Cellulose Gum (and) Xanthan Gum gave a low yield stress value, not sufficient. The premix Cellulose Gum (and) Carrageenan (and) Ceratonia siliqua (Carob) Gum (and) Glucose had no measurable effect. The premix Cellulose Gum (and) Xanthan Gum (and) Inulin (and) Cellulose (and) Glucose (and) Fructose had no measurable effect.

However, it was surprisingly observed that a combination of 2.5% of the premix Cellulose Gum (and) Carrageenan (and) Ceratonia siliqua (Carob) Gum (and) Glucose, and 1% of the premix Cellulose Gum (and) Xanthan Gum (and) Inulin (and) Cellulose (and) Glucose (and) Fructose, in water, provides a solution with a high yield stress value; wherein % are in wt % of the aqueous formulation.

It was decided to further investigate the combination of 2.5% of the premix Cellulose Gum (and) Carrageenan (and) Ceratonia siliqua (Carob) Gum (and) Glucose, and 1% of the premix Cellulose Gum (and) Xanthan Gum (and) Inulin (and) Cellulose (and) Glucose (and) Fructose, and see if it can have a stabilizing effect in a cosmetic formulation. A cosmetic emulsion as disclosed in Example 2 was selected for this test.

### Example 2: Cosmetic Formulation with a Synthetic Dispersion Stabilizing Agent

TABLE 2
Chassis of a cosmetic emulsion

| Ingredient's function (INCI) | wt % |
|---|---|
| Humectant (Glycerin) | 17 |
| Emollient | 3 |
| Viscosity Increasing Agent | 1.5 |
| Dispersion stabilizing agent (Carbomer) | 0.1 |
| Emulsifier | 0.5 |
| Petrolatum, pH Adjuster, preservative | 2.5 |
| Skin Conditioner | 1.55 |
| Solvent (Water) | q.s. for 100% |

The dispersion stabilizing agent in the selected cosmetic emulsion is a synthetic Carbomer (Tradename “Carbopol Ultrez 10”). Viscosity increasing agent includes an alkyl alcohol and 0.5% (wt % of the cosmetic emulsion) of Sodium Polyacrylate (Tradename “Cosmedia SP” from BASF).

### Example 3: Dispersion Stabilizing Agents in a Cosmetic Product Formulation

The synthetic dispersion stabilizing agent (Carbomer) present in the cosmetic emulsion disclosed in Example 2 was removed and replaced by a combination of the two premixes identified in example 1, namely the premix Cellulose Gum (and) Carrageenan (and) Ceratonia siliqua (Carob) Gum (and) Glucose (tradename “Sucraclear V2”), and the premix Cellulose Gum (and) Xanthan Gum (and) Inulin (and) Cellulose (and) Glucose (and) Fructose (tradename “preBIULIN C90”). Water content was adjusted to compensate for the difference in wt %.

Several combinations of the two premixes were tested and the results are presented in Table 3.

Sodium Polyacrylate may have a dispersion stabilizing effect in addition to its primary function as a viscosity increasing agent. Trial 14 and 15 were conducted to clarify the role of Sodium Polyacrylate in the dispersion stabilization.

TABLE 3
Chassis of a cosmetic emulsion according to Example 2 with alternative dispersion stabilizing agents.

| | | Cellulose Gum | Cellulose Gum (and) | | | | |
|---|---|---|---|---|---|---|---|
| | | (and) Carrageenan | Xanthan Gum (and) | | | | |
| | | (and) Ceratonia | Inulin (and) Cellulose | | Yield | | |
| | | Siliqua (Carob) | (and) Glucose (and) | Sodium | stress | Yield | Flow/ |
| | Carbomer | Gum (and) Glucose | Fructose | Polyacrylate | (Pa) | risk | break |
| Trial 11 | 0.10% | — | — | 0.50% | 36.6 | 0.130 | 0.927 |
| (comparative) | | | | | | | |
| Trial 12 | — | 2.50% | 1.00% | 0.50% | — | — | — |
| (inventive) | | | | | | | |
| Trial 13 | — | 2.50% | 0.50% | 0.50% | 238.28 | 0.694 | 0.828 |
| (inventive) | | | | | | | |
| Trial 14 | — | 2.50% | 0.50% | — | 95.9 | 0.989 | 0.747 |
| (inventive) | | | | | | | |
| Trial 15 | — | 1.00% | 0.50% | — | 0 | 2.164 | 0.438 |
| (comparative) | | | | | | | |

Trial 11 corresponded to the chassis disclosed in Example 2, it served as a benchmark emulsion having acceptable stability but comprising a synthetic dispersion stabilizing agents (Carbomer).

Trial 12 used the combination of the premix Cellulose Gum (and) Carrageenan (and) Ceratonia siliqua (Carob) Gum (and) Glucose, and of the premix Cellulose Gum (and) Xanthan Gum (and) Inulin (and) Cellulose (and) Glucose (and) Fructose identified in trial 10 (example 1), however the viscosity (yield stress) of the emulsion was better (higher) than expected and could not be recorded.

Trial 13 and Trial 14 contained reduced amount of the premix Cellulose Gum (and) Xanthan Gum (and) Inulin (and) Cellulose (and) Glucose (and) Fructose but still showed very good yield stress values even without Sodium Polyacrylate (Trial 14). Yield risk values were below 1, indicating a solid-like behavior. The flow/break values (shear thinning index) were high enough and compatible with an acceptable spreadability.

Trial 15 was a conducted with a reduced amount of the premix Cellulose Gum (and) Carrageenan (and) Ceratonia siliqua (Carob) Gum (and) Glucose, compared to Trial 14. The viscosity (yield stress) of the emulsion dropped dramatically and the emulsion was not stable.

These trials demonstrated that the combination of the natural ingredients identified in the present disclosure could stabilize a cosmetic formulation and serve as an alternative to a synthetic dispersion stabilizing agent such as Carbomer. This surprising effect could not have been anticipated especially considering the negative results of trials 4 and 5.

1. A dispersion stabilizing agent, comprising:
about 65% or more of cellulose gum,
about 5% to about 25% of carrageenan,
about 5% to about 25% of carob gum,
about 0.1% to about 4% of xanthan gum, and
about 0.1% to about 4% of inulin,
in wt % of the dispersion stabilizing agent.

2. The dispersion stabilizing agent of claim 1, wherein the dispersion stabilizing agent further comprises at least one carbohydrate selected from a group consisting of cellulose, glucose, fructose, or combinations thereof.

3. The dispersion stabilizing agent of claim 1, wherein the dispersion stabilizing agent is a blend of a first premix and a second premix, and wherein
the first premix comprises: cellulose gum, carrageenan and carob gum, and
the second premix comprises: cellulose gum, xanthan gum and inulin.

4. The dispersion stabilizing agent of claim 3, wherein the first premix comprises:
about 75% or more of cellulose gum,
about 10% to about 15% of carrageenan; and
about 10% to about 15% of carob gum, in wt % of the first premix.

5. The dispersion stabilizing agent of claim 3, wherein the second premix comprises:
about 50% or more of cellulose gum,
about 5% to about 10% of xanthan gum, and
about 5% to about 10% of inulin, in wt % of the second premix.

6. The dispersion stabilizing agent of claim 3, wherein the ratio between the first premix and the second premix is from about 2.1:1 to about 10:1.

7. The dispersion stabilizing agent of claim 3, wherein
the first premix comprises about 75% or more of cellulose gum, about 10% to about 15% of carrageenan; and about 10% to about 15% of carob gum, in wt % of the first premix, and
the second premix comprises about 50% or more of cellulose gum, about 5% to about 10% of xanthan gum, and about 5% to about 10% of inulin, in wt % of the second premix, and
the ratio between the first premix and the second premix is from about 2.1:1 to about 10:1.

8. A cosmetic formulation comprising the dispersion stabilizing agent of claim 1.

9. The cosmetic formulation of claim 8, wherein the dispersion stabilizing agent comprises a blend of a first premix and a second premix and wherein
the first premix comprises: cellulose gum, carrageenan and carob gum, and
the second premix comprises: cellulose gum, xanthan gum and inulin.

10. The cosmetic formulation of claim 8, wherein the amount of the dispersion stabilizing agent is from about 0.1% or more to about 5% or less, in wt % of the cosmetic formulation.

11. The cosmetic formulation of claim 8, wherein the formulation has a yield stress of more than about 30 Pa.

12. The cosmetic formulation of claim 8, wherein the formulation is an emulsion or a suspension.

13. A method of increasing the stability of a cosmetic emulsion, the method comprising combining the dispersion stabilizing agent of claim 1, with a cosmetic emulsion.

14. A method of increasing the stability of a cosmetic suspension, the method comprising combining the dispersion stabilizing agent of claim 1, with a cosmetic suspension.
